(19) United States
(12) Patent Application Publication
(10) Pub. No.: US 2026/0317808 A1
(43) Pub. Date: Oct. 15, 2026

(54) QUANTITATIVE ASSESSMENT OF RNA ENCAPSULATION

(71) Applicant: SANOFI PASTEUR INC., Swiftwater, PA (US)

(72) Inventors: Adrien NOUGAREDE, Villeurbanne (FR); Charlène VALADON, Paris (FR)

(21) Appl. No.: 19/101,175

(22) PCT Filed: Aug. 4, 2023
(86) PCT No.: PCT/EP2023/071712

§ 371 Date: Feb. 4, 2025

(30) Foreign Application Priority Data
Aug. 4, 2022 (EP) 22315177.0

(51) Int. Cl. C12Q 1/68; A61K 9/51

(52) U.S. Cl. CPC C12Q 1/68; A61K 9/5192

(57) ABSTRACT

The present invention relates to methods of determining the efficiency of RNA encapsulation in lipid nanoparticles (LNPs). In some embodiments, methods according to the present invention comprise a step a) of contacting a sample comprising RNA encapsulated in LNPs with a first fluorophore and a second fluorophore, thereby forming fluorophore-RNA complexes, and a step b) of detecting the fluorescence signals of the complexed first and second fluorophore, wherein the first fluorophore permeates the LNPs and wherein the second fluorophore does not permeate the LNPs.

### FIELD

The present invention relates to the field of RNA analysis and more particularly to the determination of RNA encapsulation efficiency.

### BACKGROUND OF THE INVENTION

Messenger RNA therapy (MRT) is becoming an increasingly important approach for the treatment of a variety of diseases. MRT involves administration of messenger RNA (mRNA) to a patient in need of the therapy for production of the protein encoded by the mRNA within the patient's body. To ensure efficient delivery of RNA to host cells in vivo, RNA is commonly encapsulated in a carrier, such as lipid nanoparticles (LNPs). Therefore, accurate characterization of the efficiency of RNA encapsulation is particularly important for determining the quality of mRNA for therapeutic applications.

RNA encapsulation efficiency is generally determined using the RiboGreen Assay, which involves a step of LNP lysis with a detergent, typically Triton X-100, to liberate encapsulated nucleic acids such that they can be detected by the RiboGreen® fluorophore. However, the use of detergent leads to foaming. This is particularly undesirable in high throughput assay conditions where sample volumes are low. In addition, to determine encapsulation efficiency, it is necessary to perform two separate measurements, on an untreated sample and a sample treated with detergent, to determine the free and total mRNA content, respectively, which allows for the calculation of the proportion of encapsulated mRNA payload.

Thus, there remains a need for improved methods of determining RNA encapsulation efficiency. Such improved methods should notably be fast, robust, cost effective, and easy to perform.

### SUMMARY OF THE INVENTION

The present invention provides improved methods for determining RNA encapsulation efficiency in lipid particles. Advantageously, the methods provide a simple, quick, and cost-effective approach for determining encapsulation efficiency. As discussed above, encapsulation efficiency is an important quality control parameter when generating RNA-based therapeutics. These methods are particularly suitable for use in quality control during or following RNA encapsulation in LNPs and batch release. Advantageously, they may be used in high-throughput and/or automated screening techniques.

A method of determining the efficiency of RNA encapsulation in lipid nanoparticles (LNPs) is provided herein, the method comprising:
a) contacting a sample comprising RNA encapsulated in LNPs with a first fluorophore and a second fluorophore, thereby forming fluorophore-RNA complexes, and
b) detecting the fluorescence signals of the complexed first and second fluorophores,
   wherein the first fluorophore permeates the LNPs and wherein the second fluorophore does not permeate the LNPs.

In certain embodiments, the first fluorophore is Quant-iT™ HS or SYBR® Green II.

In certain embodiments, the second fluorophore is RiboGreen® or SYBR® Gold.

In certain embodiments, the RNA is from 10 to 50000 nucleotides in length.

In certain embodiments, the RNA is from 300 to 10000 nucleotides in length.

In certain embodiments, the RNA is from 500 to 5000 nucleotides in length.

In certain embodiments, the RNA is double-stranded RNA or single-stranded RNA.

In certain embodiments, the RNA comprises mRNA, miRNA, shRNA, rRNA, tRNA, snRNA, snoRNA, asRNA, siRNA, aiRNA, gRNA, and/or piRNA.

In certain embodiments, the first fluorophore and the second fluorophore are added to the sample simultaneously.

In certain embodiments, the first fluorophore and the second fluorophore are added to the sample sequentially.

In certain embodiments, RNA is present at a final concentration of at least 0.25 μg/mL, optionally at a final concentration of at least 1 μg/mL. In certain embodiments, RNA is present at a final concentration of comprised between 0.25 and 10 μg/mL.

In certain embodiments, the ratio of the first fluorophore to the second fluorophore is 2:1.

In certain embodiments, the first fluorophore is present at a final concentration of 0.5×.

In certain embodiments, the second fluorophore is present at a final concentration of 0.25×.

In certain embodiments, the method further comprises determining the ratio of the second fluorescent signal to the first fluorescent signal (i.e., ratio of the fluorescent signal of second fluorophore to the fluorescent signal of first fluorophore).

In certain embodiments, the method comprises generating a standard curve for the first and second fluorescent signals.

In certain embodiments, the method comprises determining the absolute amount of encapsulated RNA by matching each fluorescent signal detected in step b) with the corresponding standard curve.

In another aspect, a method of manufacturing LNPs encapsulating RNA is provided, the method comprising:
a) encapsulating RNA in LNPs, and
b) determining efficiency of RNA encapsulation in LNPs according to the method provided herein.

In certain embodiments, the RNA is mRNA.

In certain embodiments, the method further comprises a step of synthesizing mRNA in vitro prior to step a).

In certain embodiments, the LNPs comprise one or more ionizable lipids, one or more helper lipids, and one or more PEG-modified lipids.

In certain embodiments, the in vitro synthesized mRNA is purified prior to encapsulation in LNPs.

In certain embodiments, the encapsulation efficiency is at least 80%.

In certain embodiments, the method is conducted before releasing a batch of LNPs encapsulating RNA.

In another aspect, a kit for determining efficiency of RNA encapsulation in LNPs is provided, the kit comprising:
a first fluorophore that permeates LNPs;
a second fluorophore that does not permeate LNPs; and
instructions for use according to the method provided herein.

In another aspect, the use of Quant-iT™ HS in quantifying LNP-encapsulated RNA is provided.

### BRIEF DESCRIPTION OF THE DRAWINGS

FIG. 1: Determination of mRNA encapsulation efficiency by the RiboGreen Assay or the method of the invention (the ‘dual RNA encapsulation’ assay). Following encapsulation in LNPs, mRNA is generally present in a sample in two different states: as non-encapsulated or ‘free’ mRNA and as mRNA that has been encapsulated in LNPs (mRNA-LNPs). ‘Total mRNA’ refers to the combination of both free and encapsulated mRNAs. The RiboGreen Assay (left panel) uses the RiboGreen® fluorophore (black circles), which labels RNA but does not permeate LNPs. Consequently, non-encapsulated mRNA can be measured with RiboGreen® alone (i.e., in TE buffer) while total mRNA can be measured when RiboGreen® is present in combination with a detergent, such as Triton, which lyses the LNPs. The principle of the present invention is based on the use of two fluorophores labeling RNA (right panel), one of which is non-permeant (here, RiboGreen®, black circles) while the other is permeant (here, Quant-iT™ HS, white circles). Advantageously, both fluorophores can be used in the same well without overlap of spectral detection or detrimental competition.

FIG. 2: Determination of LNP permeability to Quant-iT™ HS. Quant-iT™ HS fluorescence emission was measured for unencapsulated (free) mRNA or encapsulated mRNA at different total mRNA concentrations.

FIG. 3: Establishment of linear regression curves for RiboGreen® and Quant-iT™ HS fluorophores when in presence of each other. Eight different concentrations of unencapsulated mRNA were labeled with both RiboGreen® and Quant-iT™ HS. Simple linear regression curves, and the corresponding equations (Y) and R², were determined from these eight points for each fluorophore. (A) RiboGreen® emission at 525 nm in the presence of Quant-iT™ HS as a function of free mRNA concentration. (B) Quant-iT™ HS emission at 673 nm in the presence of RiboGreen® as a function of free mRNA concentration.

FIG. 4: Determination of RiboGreen® and Quant-iT™ HS emissions ratio for the same concentration of labeled mRNA. The RiboGreen® (R) fluorophore does not permeate LNPs; thus, it labelled only non-encapsulated mRNA. The Quant-iT™ HS (Q) fluorophore permeates LNPs; thus, it labelled both encapsulated and non-encapsulated mRNA. The Coefficient of Fluorescence (Cf) between RiboGreen® and Quant-iT™ HS was determined for different concentrations (x) of mRNA labeled with a mixture of RiboGreen® and Quant-iT™ HS. Several factors of dilution of the mRNA-LNPs were used to modulate x-R and x-Q labeled mRNA, allowing the Cr for different x to be calculated.

FIG. 5: Determination of encapsulation efficiency without standard RNA curve. The percentage of free mRNA, and thus encapsulation efficiency, can be calculated from the ratio of the RiboGreen® and Quant-iT™ HS emissions in the same well as illustrated. The Cr between RiboGreen® and Quant-iT™ HS for the same concentration of labeled mRNA was experimentally determined to be constant (0.01).

FIG. 6: Evaluation of the SYTO®17 fluorophore. (A-B) Standard mRNA concentration range for RiboGreen® fluorophore alone (A) or with SYTO® 17 in the same well (B). Five different concentrations of free mRNA were labeled with RiboGreen® (0.5×) in TE buffer with or without SYTO® 17 (1 μM). The simple linear regression curves, and the corresponding equations (Y) and R², were determined from the 5 points of the standard mRNA dilution. (C) Determination of LNP permeability to SYTO® 17. SYTO® 17 fluorescence emission was measured for free mRNA or encapsulated mRNA at different total mRNA concentrations.

FIG. 7: Evaluation of the SYBR® Green II and SYBR® Gold fluorophores. SYBR® Green II and SYBR® Gold fluorophore permeability to LNPs was evaluated to determine if these fluorophores could be suitable for use in the dual RNA encapsulation assay. (A) When using SYBR® Green II, the emission curves obtained for free mRNA and encapsulated mRNA were identical, indicating that this fluorophore permeates LNPs. (B) When using SYBR® Gold, an emission curve was obtained for free mRNA, with almost no fluorescence emissions detected for encapsulated mRNA, indicating that this fluorophore does not permeate LNPs. The simple linear regression curves determined when labeling mRNA with SYBR® Green II or SYBR® Gold indicate that these fluorophores may be utilized in the dual RNA encapsulation assay in combination with compatible fluorophores.

FIG. 8: Automation of Dual RNA encapsulation assay: validation of the method with two different spectrophotometers. Encapsulation efficiency was determined using an automated system and with two different spectrophotometers: Cytation 7 and Spectramax i3. The results were shown to be accurate independently of the spectrophotometer used and also illustrate that method according to the invention is automatable.

### DETAILED DESCRIPTION OF THE INVENTION

The present disclosure is directed, inter alia, to methods of determining RNA encapsulation efficiency.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

It is to be noted that the term “a” or “an” entity refers to one or more of that entity; for example, “a nucleotide sequence” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure.

Units, prefixes, and symbols are denoted in their International System of Units (SI) accepted form. Numeric ranges are inclusive of the numbers defining the range. The headings provided herein are not limitations of the various aspects of the disclosure. Accordingly, the terms defined immediately below are more fully defined by reference to the specification in its entirety.

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or 0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

As used herein, the term “nucleic acid” or “nucleic acid molecule” refers to a polynucleotide chain comprising individual nucleic acid residues. A “nucleic acid” encompasses single and/or double-stranded DNA and/or cDNA, as well as single and/or double-stranded RNA. Furthermore, a “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. Nucleic acid sequences that encode proteins and/or RNA may include introns. Nucleic acid may be of any origin, e.g., viral, bacterial, archae-bacterial, fungal, ribosomal, eukaryotic or prokaryotic. Nucleic acid may be purified from natural sources (e.g., from any biological sample and any organism, tissue, cell, or sub-cellular compartment), produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. In some embodiments, a nucleic acid comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyladenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the nucleic acid is composed of “unmodified nucleic acids,” meaning nucleic acids (e.g., polynucleotides and residues, including nucleotides and/or nucleosides) that have not been chemically modified. In some embodiments, the nucleic acid comprises at least one chemical modification. In some embodiments, the nucleic acid is RNA. In some embodiments, the nucleic acid is mRNA.

As used herein, the term “messenger RNA (mRNA)” refers to a polynucleotide that encodes at least one peptide, polypeptide or protein. mRNA may contain one or more coding and non-coding regions. A coding region is alternatively referred to as an open reading frame (ORF). Non-coding regions in mRNA include the 5′ cap, 5′ untranslated region (UTR), 3′ UTR, and a polyA tail.

mRNA as used herein encompasses both modified and unmodified RNA. In some embodiments, the mRNA disclosed herein may contain one or more modifications that typically enhance RNA stability. Exemplary modifications can include backbone modifications, sugar modifications, or base modifications. In some embodiments, the disclosed mRNA may be synthesized from naturally occurring nucleotides and/or nucleotide analogues (modified nucleotides) including, but not limited to, purines (adenine (A) and guanine (G)) or pyrimidines (thymine (T), cytosine (C), and uracil (U)). In certain embodiments, the disclosed mRNA may be synthesized from modified nucleotide analogues or derivatives of purines and pyrimidines, such as, e.g., 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxy acetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, p-D-mannosyl-queosine, phosphoramidates, phosphorothioates, peptide nucleotides, methylphosphonates, 7-deazaguanosine, 5-methylcytosine, and inosine.

In some embodiments, the mRNA may comprise at least one chemical modification including, but not limited to, pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the chemical modification is selected from pseudouridine, N1-methylpseudouridine, 5-methylcytosine, 5-methoxyuridine, and any combination thereof. In some embodiments, the chemical modification comprises N1-methylpseudouridine.

In some embodiments, at least 20%, at least 30%, at least 40%, at least 50%, at least 60%, at least 70%, at least 80%, at least 85%, at least 90%, at least 95%, or 100% of the uracil nucleotides in the mRNA are chemically modified. In some embodiments, at least 20%, at least 30%, at least 40%, at least 50%, at least 60%, at least 70%, at least 80%, at least 85%, at least 90%, at least 95%, or 100% of the uracil nucleotides in the ORF are chemically modified.

Where appropriate, e.g., in the case of chemically synthesized molecules, mRNA can comprise nucleoside analogs such as analogs having chemically modified bases or sugars, backbone modifications, etc. In some embodiments, an mRNA is or comprises natural nucleosides (e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, pseudouridine, and 5-methylcytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In addition to messenger RNA, several non-coding types of RNA exist which may be involved in regulation of transcription and/or translation, and immunostimulation. Within the present invention the term “RNA” further encompasses any type of single stranded (ssRNA) or double stranded RNA (dsRNA) molecule known in the art, such as viral RNA, retroviral RNA and replicon RNA, messenger RNA (mRNA), microRNA (miRNA), small hairpin RNA (shRNA), ribosomal RNA (rRNA), transfer RNA (tRNA), small nuclear RNA (snRNA), small nucleolar RNA (snoRNA), antisense RNA (asRNA), small interfering RNA (siRNA), asymmetrical interfering RNA (aiRNA), CRISPR/Cas9 guide RNA (gRNA), Piwi-interacting RNA (piRNA), dicer-substrate RNA, ribozymes, aptamers, riboswitches, immunostimulating RNA, and long non-coding RNA (lncRNA), and any combination thereof.

In one embodiment, the RNA is double-stranded RNA. In another embodiment, the RNA is single-stranded RNA. In cases where RNA is single stranded, it may further comprise one or more secondary structures, such as hairpins. In one embodiment, RNA is circular RNA (circRNA). In one embodiment, RNA is linear RNA. In one embodiment, the RNA may be any type of RNA provided herein. In one embodiment, the RNA is selected from mRNA, miRNA, shRNA, rRNA, tRNA, snRNA, snoRNA, asRNA, siRNA, aiRNA, gRNA, piRNA and any combination thereof. In one embodiment, the RNA is mRNA. In one embodiment, the RNA is miRNA. In one embodiment, the RNA is siRNA. In one embodiment, the RNA is a combination of mRNA and a second type of RNA, such as siRNA or gRNA. In one embodiment, the mRNA is synthesized in vitro.

As used herein, a “lipid nanoparticle (LNP)” is a composition comprising one or more lipids. Lipids present in an LNP may include one or more cationic/ionizable, PEGylated, helper, or other lipids, such as phospholipids. LNPs are typically sized on the order of micrometers or smaller and may include a lipid bilayer. Lipid nanoparticles, as used herein, unless otherwise specified, encompass lipid nanoparticles (LNPs), liposomes (e.g., lipid vesicles), and lipoplexes. In some embodiments, an LNP may be a liposome having a lipid bilayer with a diameter of 500 nm or less. Typically, LNPs are in the shape of a hollow sphere, encapsulating an aqueous compartment. The “lipid component” is that component of an LNP that includes one or more lipids. Typically, an LNP can be formed by mixing one or more lipids or by mixing one or more lipids and polymer(s). An LNP may notably contain ionizable/cationic lipid(s) and optionally non-cationic lipid(s), optionally cholesterol-based lipid(s), and/or optionally PEGylated lipid(s). A “non-cationic lipid” or “helper lipid” refers to any neutral, zwitterionic, or anionic lipid. A “PEG lipid” or “PEGylated lipid” refers to a lipid comprising a polyethylene glycol component.

As used herein, “encapsulation” and its grammatical equivalents refer to the process of confining nucleic acid within a nanoparticle. Encapsulation may notably be complete, substantial, or partial. Nucleic acid may be located in an aqueous phase within the liposome or integrated into a lipid layer. As used herein, an “empty” LNP may refer to a nanoparticle that is substantially free of a nucleic acid. As used herein, an “empty” LNP may refer to a nanoparticle that consists substantially of only lipid components. Nucleic acid that is not encapsulated in an LNP is referred to herein as “unencapsulated,” “non-encapsulated, or “free” nucleic acid.

As used herein, “encapsulation efficiency” and “efficiency of encapsulation” refer to the amount of a nucleic acid that is integrated into the internal structure of an LNP (i.e., that is not accessible to hydrophilic solvents or molecules when the LNP is intact), relative to the initial total amount of nucleic acid used in the preparation of an LNP. As an example, if 95 mg of nucleic acid are encapsulated in a LNP out of a total 100 mg of nucleic acid initially provided to the composition, the encapsulation efficiency may be given as 95%. Total initial nucleic acid may notably be determined by adding the amount of encapsulated nucleic acid to the amount of non-encapsulated nucleic acid in a given sample.

As used herein, the term “sample comprising nucleic acid encapsulated in LNPs” denotes a sample that comprises nucleic acid, e.g., RNA or mRNA, encapsulated in LNPs. In some embodiments, the sample comprising nucleic acid encapsulated in LNPs comprises a mixture of nucleic acid encapsulated in LNPs and of unencapsulated nucleic acid. The sample comprising nucleic acid (e.g., RNA or mRNA) encapsulated in LNPs is for example a batch of RNA-LNPs or mRNA-LNPs as obtained by a method of manufacturing LNPs encapsulating RNA or mRNA.

As used herein, the term “RNA-LNP” refers to RNA encapsulated in LNP. Similarly, “mRNA-LNP” refers to mRNA encapsulated in LNP.

As used herein, the term “labeled” refers to attachment of a detectable signal, agent or moiety, such as a fluorophore, to a molecule, such as a nucleic acid molecule.

A “fluorophore” or “fluorescent dye” as used herein refers to a chemical group that absorbs light at a particular wavelength (excitation frequency) and subsequently emits light of a longer wavelength (emission frequency), i.e., it fluoresces. Fluorophores may contain substituents that alter the solubility, spectral properties, or physical properties of the fluorophore. A fluorophore may be conditionally fluorescent, i.e., the level of fluorescence increases when the fluorophore binds to its target as compared to the level of fluorescence when the fluorophore is in its unbound form. Numerous fluorophores are known to those skilled in the art and include, but are not limited to, a coumarin, a cyanine dye, a phenanthridinium dye, a bisbenzimide dye, a bisbenzimidazole dye, an acridine dye, a chromomycinone dye, a benzofuran dye, a quinoline dye, a quinazolinone dye, an indole dye, a pyrene dye, a merocyanine dye, a benzocyanine dye, a penzopyrilium dye, a benzazole dye, a borapolyazaindacene dye, a xanthene dye including fluoroscein, rhodamine, or rhodol, as well as other fluorophores described in The Molecular Probes® Handbook: A Guide to Fluorescent Probes and Labeling Technologies (11th edition, 2010), and US 2005/0208534.

The term “detectably distinct” as used herein refers to a signal that is distinguishable or separable by a physical property either by observation or by instrumentation. For example, a fluorophore is readily distinguishable from another fluorophore in a sample, as well as from additional materials that are optionally present, by spectral characteristics, i.e., excitation and emission spectra.

As used herein, the terms “sensitivity range” or “sensitivity scale” refers to the range of RNA concentrations for which a given labeling fluorophore gives a linear curve of emission.

“Permeability” as used herein refers to the material properties that enable one or more substances to pass through a material. “Selectively permeable” refers to the material properties that allow specific substances (e.g., fluorophores) to pass through the material while preventing other substances from passing through the material. In the present context, the term “permeate” refers to the ability of a substance (i.e., a fluorophore) to penetrate or pass through a lipid structure, such as the lipid component of an LNP. The ability of a fluorophore to permeate (or not) LNPs can be easily determined by comparing the level of fluorescence detected between two samples: a first sample comprising free mRNA and LNP encapsulated mRNA (mRNA-LNP) with known concentrations of free mRNA/mRNA-LNP (e.g. as can be determined by the RiboGreen assay), and a second sample which has a concentration of free mRNA identical to either the concentration of free mRNA in the first sample, or to the total concentration of mRNA (free mRNA+mRNA-LNP) in the first sample. If fluorescence levels are the same for the first sample and for a second sample having a concentration of free mRNA identical to the total concentration of mRNA in the first sample, then the fluorophore is permeating. If fluorescence levels are the same for the first sample and for a second sample having a concentration of free mRNA identical to the concentration of free mRNA in the first sample, then the fluorophore is non-permeating.

As used herein, the term “contacting” refers to the mixing of two or more components such that those components are capable of interacting (e.g., contacting an RNA-LNP with fluorophores). The two or more components may be incubated for any time sufficient to produce a desired effect (e.g., such that they form a complex).

As used herein, the term “control” refers to a standard against which results may be compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

As used herein, the term “kit” refers to any delivery system for delivering materials. Such delivery systems may include systems that allow for the storage, transport, or delivery of various diagnostic or therapeutic reagents (e.g., oligonucleotides, antibodies, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a sub-portion of the total kit components. The containers may be delivered to the intended recipient together or separately. For example, a first container may contain an enzyme for use in an assay, while a second container contains oligonucleotides. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contains a sub-portion of the total kit components are included in the term “fragmented kit.” In contrast, a “combined kit” refers to a delivery system containing all of the components in a single container (e.g., in a single box housing each of the desired components). The term “kit” includes both fragmented and combined kits.

As described in detail below, the present invention is based on the use of two fluorophores having different permeability profiles to LNPs and that bind to nucleic acid. Specifically, a first fluorophore permeates the LNP encapsulating the nucleic acid (e.g., an mRNA-LNP) while a second fluorophore does not permeate the LNP. The first fluorophore forms complexes with encapsulated and unencapsulated nucleic acid that may be present in the sample, while the second fluorophore forms complexes with unencapsulated nucleic acid that may be present in the sample. Detection of fluorescence emissions of the two fluorophores ultimately allows for encapsulation efficiency to be established. Advantageously, the method of the invention reduces the number of samples needed for determining encapsulation efficiency by at least two-fold, as both fluorescent measurements can be performed on a single sample. In addition, the method can be used in high-throughput screening, as foaming is no longer an issue in the absence of detergent. Thus, the present invention provides a simple, reliable, and efficient quantitative or semi-quantitative approach for assessing RNA encapsulation efficiency. The present invention is particularly useful for quality control during manufacturing and for characterization of encapsulated nucleic acid, such as mRNA, as a pharmaceutical ingredient in final therapeutic products.

In one aspect, the present invention provides a method for determining the efficiency of nucleic acid encapsulation in LNPs, said method comprising the steps of:
a) contacting a sample comprising nucleic acid encapsulated in LNPs with a first fluorophore and a second fluorophore, thereby forming fluorophore-nucleic acid complexes, and
b) detecting the fluorescence signals of the complexed first and second fluorophore,
   wherein the first fluorophore permeates the LNPs and wherein the second fluorophore does not permeate the LNPs.

While encapsulation efficiency is desirably high (e.g., close to 100%), e.g., for encapsulated nucleic acid that is to be used in pharmaceutical products, when using the method of the present invention, an encapsulation efficiency ranging from 0 to 100% may be determined. Thus, in some embodiments, the encapsulation efficiency that is determined is from 0 to 100%. In some embodiments, the encapsulation efficiency is at least about 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency is at least 80%. In some embodiments, the encapsulation efficiency is at least 90%. In some embodiments, the encapsulation efficiency is at least 91%. In some embodiments, the encapsulation efficiency is at least 92%. In some embodiments, the encapsulation efficiency is at least 93%. In some embodiments, the encapsulation efficiency is at least 94%. In some embodiments, the encapsulation efficiency is at least 95%. In some embodiments, the encapsulation efficiency is at least 96%. In some embodiments, the encapsulation efficiency is at least 97%. In some embodiments, the encapsulation efficiency is at least 98%. In some embodiments, the encapsulation efficiency is at least 99%. In some embodiments, the encapsulation efficiency is from 80 to 100%.

The total concentration of nucleic acid (i.e., both unencapsulated and encapsulated) is such that it falls within the sensitivity range of the fluorophores used in the method of the invention. In one embodiment, nucleic acid is present at a final concentration of at least 0.25 μg/mL. In one embodiment, nucleic acid is present at a final concentration comprised between 0.25 and 10 μg/mL. In one embodiment, nucleic acid is present at a final concentration comprised between 0.25 and 5 μg/mL. In one embodiment, nucleic acid is present at a final concentration comprised between 0.25 and 1.25 μg/mL. In one embodiment, nucleic acid is present at a final concentration comprised between 0.25 and 1 μg/mL. A sample comprising nucleic acid encapsulated in LNPs may be subjected to one or more dilutions to provide a nucleic acid concentration that falls within the sensitivity range of the fluorophores.

The nucleic acid encapsulated in the LNPs may be any type of nucleic acid as provided herein. Encapsulated nucleic acid molecules (e.g., RNA) may be of any length. In one embodiment, a nucleic acid molecule is at least about 10 nucleotides (nt) in length. In one embodiment, a nucleic acid molecule is at least about 20 nt in length. In one embodiment, a nucleic acid molecule is at least about 50 nt in length. In one embodiment, a nucleic acid molecule is at least about 100 nt in length. In one embodiment, a nucleic acid molecule is at least about 500 nt in length. In one embodiment, a nucleic acid molecule is from about 10 to 50000 nt in length. In one embodiment, a nucleic acid molecule is from about 20 to 25000 nt in length. In one embodiment, a nucleic acid molecule is from about 300 to 10000 nt in length. In one embodiment, a nucleic acid molecule is from about 300 to 10000 nt in length. In one embodiment, a nucleic acid molecule is from about 400 to 8000 nt in length. In one embodiment, a nucleic acid molecule is from about 500 to 5000 nt in length. In some embodiments, the LNP comprises 1-20, optionally 5-10 or 6-8, nucleic acid molecules.

The first fluorophore provided in the context of the method permeates LNPs. In one embodiment, the first fluorophore is the Quant-iT™ High Sensitivity reagent, also referred to herein as Quant-iT™ HS. The Quant-iT™ HS reagent is composed of the RiboRed fluorophore and can therefore alternatively be referred to herein as “RiboRed”. In one embodiment, the excitation spectra of Quant-iT™ HS is comprised within the range of 640 nm to 648 nm. In one embodiment, the excitation spectra of Quant-iT™ HS is 644 nm. In one embodiment, excitation is performed with a bandwidth of 9 nm (e.g., 644 nm+/−4.5 nm). In one embodiment, the emission spectra of Quant-iT™ HS is comprised within the range of 666 nm to 680 nm. In one embodiment, the emission spectra of Quant-iT™ HS is 673 nm. In one embodiment, emission spectra is collected with a bandwidth of 15 nm (e.g. 673 nm+/−7.5 nm). In one embodiment, the excitation and emission spectra of Quant-iT™ HS are 644 and 673 nm, respectively. In another embodiment, the first fluorophore is the SYBR® Green II fluorophore. In one embodiment, the excitation spectra of SYBR® Green II is comprised within the range of 491 nm to 499 nm. In one embodiment, the excitation spectra of SYBR® Green II is 495 nm. In one embodiment, excitation is performed with a bandwidth of 9 nm (e.g., 495 nm+/−4.5 nm). In one embodiment, the emission spectra of SYBR® Green II is comprised within the range of 513 nm to 527 nm. In one embodiment, the emission spectra of SYBR® Green II is 520 nm. In one embodiment, emission spectra is collected with a bandwidth of 15 nm (e.g., 520 nm+/−7.5 nm). In one embodiment, the excitation and emission spectra of SYBR® Green II are 495 and 520 nm, respectively.

The second fluorophore provided in the context of the method does not permeate LNPs. The skilled person will furthermore readily understand that the second fluorophore should be detectably distinct from the first fluorophore, to ensure that the fluorescence of each fluorophore can be measured from a single sample. In one embodiment, the first fluorophore is a cyanine dye. In one embodiment, the second fluorophore is a cyanine dye. In one embodiment, the second fluorophore is RiboGreen® (see e.g., Jones et al., Analytical Biochemistry. (1998) 265:368-374). RiboGreen® is detectably distinct from Quant-iT™ HS. In one embodiment, RiboGreen® excitation may be performed at 485±10 nm. In one embodiment, RiboGreen® excitation may be performed at 486±5 nm. In one embodiment, the excitation spectra of RiboGreen® is comprised within the range of 475 nm to 495 nm. In one embodiment, the excitation spectra of RiboGreen® is comprised within the range of 470 nm to 491 nm. In one embodiment, the excitation spectra of RiboGreen® is 485 nm. In one embodiment, excitation is performed with a bandwidth of 9 nm (e.g., 485 nm+/−4.5 nm). In one embodiment, RiboGreen® fluorescence emission may be collected at 530±15 nm. In one embodiment, the emission spectra of RiboGreen® is comprised within the range of 515 nm to 545 nm. In one embodiment, the emission spectra of RiboGreen® is 525 nm. In one embodiment, emission spectra is collected with a bandwidth of 15 nm (e.g. 525 nm+/−7.5 nm). In one embodiment, the excitation and emission spectra of RiboGreen® are 485 nm and 525 nm, respectively. In another embodiment, the second fluorophore is SYBR® Gold. SYBR® Gold is also referred to as [2-(4-{[diethyl(methyl)ammonio]methyl}phenyl)-6-methoxy-1-methyl-4-{[(2Z)-3-methyl-1,3-benzoxazol2-ylidene]methyl}quinolin-1-ium]. In one embodiment, the excitation spectra of SYBR® Gold is comprised within the range of 491 to 499 nm. In one embodiment, the excitation spectra of SYBR® Gold is 495 nm. In one embodiment, excitation is performed with a bandwidth of 9 nm (e.g., 495 nm+/−4.5 nm). In one embodiment, the emission spectra of SYBR® Gold is 537 nm. In one embodiment, emission spectra is collected with a bandwidth of 15 nm (e.g. 537 nm+/−7.5 nm). In one embodiment, the emission spectra of SYBR® Gold is comprised within the range of 530 to 544 nm. In one embodiment, the excitation and emission spectra of SYBR® Gold are 495 and 537 nm, respectively.

As will be readily understood by the skilled person, for accurate determination of encapsulation efficiency, fluorophores should not generate non-specific fluorescence, e.g., resulting from interactions of the fluorophore with the lipid component of the LNPs. In some embodiments, a control sample that does not comprise nucleic acid may be used to determine baseline fluorescence. This baseline may be subtracted from fluorescence emissions detected in corresponding sample(s) comprising LNPs encapsulating nucleic acid.

In some embodiments, one or both fluorophores are conditionally fluorescent upon binding with nucleic acid.

To ensure that the fluorescence signals from the complexed first and second fluorophores can be individually identified, the fluorescent signals of the complexed first and second fluorophores should be detectably distinct. In one embodiment, the fluorophores used in the method do not have overlapping excitation and emission spectra. In cases where spectra may overlap, an appropriate cutoff may be used to distinguish between the signals.

To ensure that the fluorescence signals from the complexed first and second fluorophores can be determined without interference, the first and second fluorophores should not compete for binding to the nucleic acid.

For accurate determination of encapsulation efficiency, the sensitivity range of said first and second fluorophores should encompass the total concentration of nucleic acid (i.e., both unencapsulated and encapsulated) present in the sample.

Fluorophore concentrations may be expressed in units (e.g., μg/mL) or as a dilution factor with regards to the initial concentration provided by a manufacturer. For example, the dilution of a 200× concentrated solution to a 1× concentration may be expressed as a dilution of 1:200. Alternatively, the fluorophore concentrations may be expressed as the final concentration used (e.g., 0.5×, 1×, etc.). In one embodiment, the first fluorophore is present at a final concentration of 0.05× to 10×, with regard to the concentration provided by the manufacturer. In one embodiment, the first fluorophore is present at a final concentration of 0.1× to 5×. In one embodiment, the first fluorophore is present at a final concentration of 0.2× to 1×. In one embodiment, the first fluorophore is present at a final concentration of 0.5×. In one embodiment, the second fluorophore is present at a final concentration of 0.05 to 10×. In one embodiment, the second fluorophore is present at a final concentration of 0.075× to 5×. In one embodiment, the second fluorophore is present at a final concentration of 0.1× to 1×. In one embodiment, the second fluorophore is present at a final concentration of 0.1× to 0.5×. In one embodiment, the second fluorophore is present at a final concentration of 0.25×. In one embodiment, the ratio of the first fluorophore to the second fluorophore is comprised within the range of 1:1 to 20:1. In one embodiment, the ratio of the first fluorophore to the second fluorophore comprised within the range of 1.5:1 to 10:1. In one embodiment, the ratio of the first fluorophore to the second fluorophore comprised within the range of 2:1 to 4:1. In one embodiment, the ratio of the first fluorophore to the second fluorophore is 4:1. In one embodiment, the ratio of the first fluorophore to the second fluorophore is 2:1.

In one embodiment, the first and second fluorophores are Quant-iT™ HS and RiboGreen®, respectively. In one embodiment, the ratio of Quant-iT™ HS to RiboGreen® is 2:1. In one embodiment, Quant-iT™ HS is provided at a final concentration of 0.5× and RiboGreen® is provided at a final concentration of 0.25×.

Prior to the measurement of fluorescence emissions, samples are contacted with the fluorophores for a quantity of time sufficient to allow fluorophore binding to nucleic acid, such that they form a complex. In one embodiment, fluorophores are incubated with a sample for more than 5 min. In one embodiment, fluorophores are incubated with a sample for more than 10 min. In one embodiment, fluorophores are incubated with a sample for 15-30 min. In one embodiment, fluorophores are incubated with a sample for 20 min. In one embodiment, fluorophores are incubated with a sample for 25 min. In one embodiment, incubation occurs in the dark. In one embodiment, incubation occurs at room temperature. In one embodiment, fluorophores are incubated with a sample for 15-30 min at room temperature in the dark.

The above-mentioned fluorophores bind to RNA. Alternative fluorophores may notably be used when the nucleic acid is not RNA. As an example, the PicoGreen® fluorophore may be envisaged when the nucleic acid is dsDNA.

In one embodiment, the first and second fluorophores are added to the sample simultaneously, i.e., in a pre-mixed solution. In another embodiment, the first and second fluorophores are added to the sample separately. In one embodiment, both fluorophores are added to the sample prior to measurement of fluorescence. In another embodiment, addition of the first fluorophore and measurement of the first fluorescent signal of said first fluorophore is followed by addition of the second fluorophore and measurement of the second fluorescent signal, or vice versa.

Controls may be used to quantitate the amount of encapsulated nucleic acid. In some embodiments, the control comprises a sample with a predetermined amount of nucleic acid. In some embodiments, the control comprises a predetermined amount of free nucleic acid. In some embodiments, the control comprises a predetermined amount of encapsulated nucleic acid. In some embodiments, the control comprises a predetermined amount of total nucleic acid. In some embodiments, the control comprises a predetermined amount of both free and encapsulated nucleic acid. In some embodiments, the control may be a predetermined amount of free RNA. In some embodiments, the control may be an RNA-LNP comprising a predetermined amount of encapsulated RNA.

Efficiency of nucleic acid (e.g., RNA or mRNA) encapsulation in lipid nanoparticles is determined based on the detected fluorescence signals of the complexed first and second fluorophores.

In some embodiments, assays are made quantitative by establishing a calibration curve (also referred to as a standard curve). In other words, encapsulation efficiency can be determined qualitatively or semi-quantitatively by comparison of the fluorescent signals detected in unknown samples with known standards or quantitatively by comparison with standard curves prepared using a number of samples of known nucleic acid concentration. For example, quantitation may be performed by making a set of standards or calibrators having a known quantity of total nucleic acid and/or a known quantity of free nucleic acid. These standards or calibrators can be serially diluted, and the resulting signal value from each tested concentration of standard or calibrator is used to generate a standard curve; plotting the concentration of standards versus the resulting signal values. Once a standard quantitative curve is established, the levels of total and free nucleic acid in a sample can be determined by plotting the resulting signal on the corresponding standard curves.

Thus, in one embodiment, the method further comprises generating a standard curve for the first and second fluorescent signals. In one embodiment, the method further comprises determining the absolute amount of encapsulated RNA by plotting each fluorescent signal that is detected in step b) with the corresponding standard curve. The amount of non-encapsulated nucleic acid (in percent) can then be determined by dividing the amount of nucleic acid detected with the second fluorophore (i.e., the fluorophore that does not permeate LNPs) by that detected with the first fluorophore (i.e., the fluorophore that permeates LNPs). The corresponding equation is illustrated below:

$$Free nucleic acid ( \% ) = \frac{Unencapsulated nucleic acid}{Total nucleic acid} \times 100$$

Encapsulation efficiency (i.e., the relative amount (in percent) of encapsulated nucleic acid) can then be determined by subtracting the quantity of free (unencapsulated) nucleic acid from 100, as illustrated in the equation below:

Encapsulation efficiency (%)=100−Free nucleic acid (%)

In some embodiments, the ratio of fluorescence between the two fluorophores may be used to directly determine encapsulation efficiency (i.e., without the need to generate standard curves). In some embodiments, a ratio of fluorescence is first established between the two fluorophores over a range of nucleic acid concentrations. By dividing the fluorescent signal of the second fluorophore by the fluorescent signal of the first fluorophore at each nucleic acid concentration, a coefficient of fluorescence (Cr) can be obtained. In some embodiments, the Cr is 0.01. In some embodiments, the fluorescent signal provided by the second fluorophore (i.e., the fluorophore that does not permeate LNPs, and that thus binds unencapsulated nucleic acid) is divided by the fluorescent signal provided by the first fluorophore (i.e., the fluorophore that permeates LNPs, and that thus binds total nucleic acid). This allows for a relative amount (in percent) of free nucleic acid to be determined. An exemplary equation is illustrated below:

$$Free nucleic acid ( \% ) = \frac{Fluorescent signal for unencapsulated nucleic acid \times 0.01 \times 100}{Fluorescent signal for total nucleic acid}$$

This equation can alternatively be expressed as:

$$Free nucleic acid ( \% ) = \frac{Fluorescent signal for unencapsulated nucleic acid}{Fluorescent signal for total nucleic acid}$$

Encapsulation efficiency (in percent) can then be determined by subtracting the quantity of free nucleic acid from 100, as described above.

In some embodiments, a sample which does not comprise any nucleic acid (i.e., a blank) may be measured, and the corresponding fluorescent signal subtracted from the signal(s) measured in a corresponding sample comprising nucleic acid. In some embodiments, the blank may comprise empty LNPs.

In some embodiments, the ratio of fluorescence is determined at two RNA-LNP dilutions which are then averaged to provide the amount of free nucleic acid (in percent). In some embodiments, the estimated final concentration of RNA in a dilution (i.e., total RNA) is comprised between 0.25 and 10 μg/mL. In some embodiments, the estimated final concentration of RNA in a dilution is comprised between 0.25 and 5 μg/mL. In some embodiments, the estimated final concentration of RNA is about 1 and about 1.25 μg/mL for the two RNA-LNP dilutions, respectively.

In some embodiments, the method provided herein is used to characterize encapsulation of a batch of RNA-LNPs. In some embodiments, the method provided herein is performed before releasing a batch of LNPs encapsulating nucleic acid. In some embodiments, the encapsulation efficiency determined according to the method provided herein is at least 80% for batch release. Thus, in one embodiment, a method for batch release comprising: a) determining the efficiency of RNA encapsulation in LNPs according to the method provided herein and b) releasing a batch of RNA-LNPs when the encapsulation efficiency is at least 80%.

The present invention further relates to a method of manufacturing LNPs encapsulating nucleic acid, comprising a step a) of encapsulating nucleic acid in LNPs, and a step b) of determining efficiency of nucleic acid encapsulation in LNPs according to the method provided herein. In one embodiment, the nucleic acid is RNA, such as mRNA, as described herein. Thus, a method of manufacturing LNPs encapsulating RNA, comprising a step a) of encapsulating RNA in LNPs, and a step b) of determining efficiency of RNA encapsulation in LNPs is according to the method provided herein is further disclosed.

In one embodiment, the LNPs comprise one or more ionizable lipids, one or more helper lipids, and one or more PEG-modified lipids.

In one embodiment, the nucleic acid molecule and/or LNP corresponds to that disclosed in US 2022/0142923, incorporated herein by reference in its entirety. In particular, the LNPs may comprise four categories of lipids: (i) an ionizable lipid; (ii) a PEGylated lipid; (iii) a cholesterol-based lipid, and (iv) a helper lipid.

### Ionizable Lipids.

An ionizable lipid facilitates mRNA encapsulation and may be a cationic lipid. A cationic lipid affords a positively charged environment at low pH to facilitate efficient encapsulation of the negatively charged mRNA drug substance.

In some embodiments, the cationic lipid is OF-02. OF-02 is a non-degradable structural analog of OF-Deg-Lin. OF-Deg-Lin contains degradable ester linkages to attach the diketopiperazine core and the doubly-unsaturated tails, whereas OF-02 contains non-degradable 1,2-amino-alcohol linkages to attach the same diketopiperazine core and the doubly-unsaturated tails (Fenton et al, Adv Mater. (2016) 28:2939; U.S. Pat. No. 10,201,618).

In some embodiments, the cationic lipid is cKK-E10 (Dong et al., PNAS (2014) 111(11):3955-60; U.S. Pat. No. 9,512,073).

In some embodiments, the cationic lipid is GL-HEPES-E3-E10-DS-3-E18-1 (2-(4-(2-((3-(Bis((Z)-2-hydroxyoctadec-9-en-1-yl)amino)propyl)disulfaneyl)ethyl)piperazin-1-yl)ethyl 4-(bis(2-hydroxydecyl)amino)butanoate), GL-HEPES-E3-E12-DS-4-E10 (2-(4-(2-((3-(bis(2-hydroxydecyl)amino)butyl)disulfaneyl)ethyl)piperazin-1-yl)ethyl 4-(bis(2-hydroxydodecyl)amino)butanoate), or GL-HEPES-E3-E12-DS-3-E14 (2-(4-(2-((3-(Bis(2-hydroxytetradecyl)amino)propyl)disulfaneyl)ethyl)piperazin-1-yl)ethyl 4-(bis(2-hydroxydodecyl)amino)butanoate), which are HEPES-based disulfide cationic lipids with a piperazine core.

Other cationic lipids that can be used include those described in Dong, supra; and U.S. Pat. No. 10,201,618.

### PEGylated Lipids

A PEGylated lipid component provides control over particle size and stability of the nanoparticle. The addition of such components may prevent complex aggregation and provide a means for increasing circulation lifetime and increasing the delivery of the lipid-nucleic acid pharmaceutical composition to target tissues (Klibanov et al., FEBS Letters (1990) 268(1):235-7). These components may be selected to rapidly exchange out of the pharmaceutical composition in vivo (see, e.g., U.S. Pat. No. 5,885,613).

### Contemplated PEGylated Lipids Include, but are not Limited to, a Polyethylene Glycol

(PEG) chain of up to 5 kDa in length covalently attached to a lipid with alkyl chain(s) of C₆-C₂₀ (e.g., C₈, C₁₀, C₁₂, C₁₄, C₁₅, or C₁₈) length, such as a derivatized ceramide (e.g., N-octanoyl-sphingosine-1-[succinyl(methoxypoly ethylene glycol)](C8 PEG ceramide)). In some embodiments, the PEGylated lipid is 1,2-dimyristoyl-rac-glycero-3-methoxypolyethylene glycol (DMG-PEG); 1,2-distearoyl-sn-glycero-3-phosphoethanolamine-polyethylene glycol (DSPE-PEG); 1,2-dilauroyl-sn-glycero-3-phosphoethanolamine-polyethylene glycol (DLPE-PEG); or 1.2-distearoyl-rac-glycero-polyethelene glycol (DSG-PEG).

In particularly exemplary embodiments, the PEG has a high molecular weight, e.g., 2000-2400 g/mol. In some embodiments, the PEG is PEG2000 (or PEG-2K). In particular embodiments, the PEGylated lipid herein is DMG-PEG2000, DSPE-PEG2000, DLPE-PEG2000, DSG-PEG2000, or C8 PEG2000.

### Cholesterol-Based Lipids

A cholesterol component provides stability to the lipid bilayer structure within the nanoparticle. In some embodiments, the LNPs comprise one or more cholesterol-based lipids. Suitable cholesterol-based lipids include, for example: DC-Choi (N,N-dimethyl-N-ethylcarboxamidocholesterol), 1,4-bis(3-N-oleylamino-propyl)piperazine (Gao et al., Biochem Biophys Res Comm. (1991) 179:280; Wolf et al., BioTechniques (1997) 23:139; U.S. Pat. No. 5,744,335), imidazole cholesterol ester (“ICE”; WO 2011/068810), [3-sitosterol, fucosterol, stigmasterol, and other modified forms of cholesterol. In some embodiments, a cholesterol-based lipid used in the LNPs is cholesterol.

### Helper Lipids

A helper lipid enhances the structural stability of the LNP and helps the LNP in endosome escape. It improves uptake and release of the mRNA drug payload. In some embodiments, a helper lipid is a neutral lipid, i.e., a lipid that does not carry a net charge in the conditions under which the composition is formulated and/or administered. In some embodiments, a helper lipid is an “anionic lipid”, i.e., a lipid that carries a net negative charge at a selected pH, such as physiological pH. In some embodiments, the helper lipid is a zwitterionic lipid, which has fusogenic properties for enhancing uptake and release of the drug payload. Examples of helper lipids are 1,2-dioleoyl-SN-glycero-3-phosphoethanolamine (DOPE); 1,2-distearoyl-sn-glycero-3-phosphocholine (DSPC); 1,2-dioleoyl-sn-glycero-3-phospho-L-serine (DOPS); 1,2-dielaidoyl-sn-glycero-3-phosphoethanolamine (DEPE); and 1,2-dioleoyl-sn-glycero-3-phosphocholine (DPOC), dipalmitoylphosphatidylcholine (DPPC), 1,2-dilauroyl-sn-glycero-3-phosphocholine (DLPC), 1,2-distearoylphosphatidylethanolamine (DSPE), and 1,2-dilauroyl-sn-glycero-3-phosphoethanolamine (DLPE).

Other exemplary helper lipids are dioleoylphosphatidylcholine (DOPC), dioleoylphosphatidylglycerol (DOPG), dipalmitoylphosphatidylglycerol (DPPG), palmito yloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a combination thereof.

In particular embodiments, the helper lipid is DOPE. In further embodiments, the LNPs comprise (i) a cationic lipid selected from OF-02, cKK-E10, GL-HEPES-E3-E10-DS-3-E18-1, GL-HEPES-E3-E12-DS-4-E10, and GL-HEPES-E3-E12-DS-3-E14; (ii) DMG-PEG2000; (iii) cholesterol; and (iv) DOPE.

### Molar Ratios of the Lipid Components

When a cationic lipid, PEGylated lipid, cholesterol-based lipid, and helper lipid are present, the molar ratio is provided as A:B:C:D, where A+B+C+D=100%. In some embodiments, the molar ratio of the cationic lipid in the LNPs relative to the total lipids (i.e., A) is 35-45% (e.g., 38-42% such as 40%). In some embodiments, the molar ratio of the PEGylated lipid component relative to the total lipids (i.e., B) is 0.25-2.75% (e.g., 1-2% such as 1.5%). In some embodiments, the molar ratio of the cholesterol-based lipid relative to the total lipids (i.e., C) is 20-35% (e.g., 27-30% such as 28.5%). In some embodiments, the molar ratio of the helper lipid relative to the total lipids (i.e., D) is 25-35% (e.g., 28-32% such as 30%). In some embodiments, the (PEGylated lipid+cholesterol) components have the same molar amount as the helper lipid. In some embodiments, the LNPs contain a molar ratio of the cationic lipid to the helper lipid that is more than 1.

In particular embodiments, the LNPs contain a cationic lipid, a PEGylated lipid, a cholesterol-based lipid, and a helper lipid at a molar ratio of 40:1.5:28.5:30. In further specific embodiments, the LNPs contain (i) OF-02, cKK-E10, GL-HEPES-E3-E10-DS-3-E18-1, GL-HEPES-E3-E12-DS-4-E10, or GL-HEPES-E3-E12-DS-3-E14; (ii) DMG-PEG2000; (iii) cholesterol; and (iv) DOPE at 40:1.5:28.5:30.

To calculate the actual amount of each lipid to be put into an LNP formulation, the molar amount of the cationic lipid is first determined based on a desired N/P ratio, where N is the number of nitrogen atoms in the cationic lipid and P is the number of phosphate groups in the mRNA to be transported by the LNP. Next, the molar amount of each of the other lipids is calculated based on the molar amount of the cationic lipid and the molar ratio selected. These molar amounts are then converted to weights using the molecular weight of each lipid.

In one embodiment, nucleic acid is encapsulated in an LNP comprising: a cationic lipid at a molar ratio between 35% and 45%, a polyethylene glycol (PEG) conjugated (PEGylated) lipid at a molar ratio between 0.25% and 2.75%, a cholesterol-based lipid at a molar ratio between 20% and 35%, and a helper lipid at a molar ratio of between 25% and 35%, wherein all the molar ratios are relative to the total lipid content of the LNP. In some embodiments, the cationic lipid is OF-02, cKK-E10, GL-HEPES-E3-E10-DS-3-E18-1, GL-HEPES-E3-E12-DS-4-E10, or GL-HEPES-E3-E12-DS-3-E14. In some embodiments, the LNP comprises a cationic lipid at a molar ratio of 40%, a PEGylated lipid at a molar ratio of 1.5%, a cholesterol-based lipid at a molar ratio of 28.5%, and a helper lipid at a molar ratio of 30%.

In some embodiments, the cationic lipid is OF-02, cKK-E10, GL-HEPES-E3-E10-DS-3-E18-1, GL-HEPES-E3-E12-DS-4-E10, or GL-HEPES-E3-E12-DS-3-E14, the PEGylated lipid is dimyristoyl-PEG2000 (DMG-PEG2000), the cholesterol-based lipid is cholesterol, and/or the helper lipid is 1,2-dioleoyl-SN-glycero-3-phosphoethanolamine (DOPE). In particular embodiments, the LNP comprises OF-02, cKK-E10, GL-HEPES-E3-E10-DS-3-E18-1, GL-HEPES-E3-E12-DS-4-E10, or GL-HEPES-E3-E12-DS-3-E14 at a molar ratio of 40%, DMG-PEG2000 at a molar ratio of 1.5%, cholesterol at a molar ratio of 28.5%, and DOPE at a molar ratio of 30%.

In some embodiments, the LNP may have a mean diameter of from about 30 nm to about 200 nm, from about 80 nm to about 150 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, or 200 nm. In some embodiments, the LNPs are substantially non-toxic. The one or more nucleic acid molecules, when present in one or more LNPs, are typically resistant to degradation with a nuclease in aqueous solution. In some embodiments, RNA-LNPs have an N/P-ratio of 1-20, 1-15, 1-10, 2-8, 2-6, or 2-4. The term “N/P ratio” refers to a molar ratio of positively charged molecular units in the cationic lipids in an LNP relative to negatively charged molecular units in the RNA encapsulated within that LNP. As such, N/P ratio is typically calculated as the ratio of moles of amine groups in cationic lipids in a LNP relative to moles of phosphate groups in RNA encapsulated within that LNP. In some embodiments, the N/P ratio is from about 1 to about 20, from about 1 to about 18, from about 1 to about 16, from about 1 to about 14, from about 1 to about 12, from about 1 to about 10, from about 1 to about 8, or from about 1 to about 6. In some embodiments, the N/P ratio is from about 2 to about 20, from about 2 to about 16, from about 2 to about 12, about 2 to about 8, or from about 2 to about 4. In some embodiments, the N/P ratio is from about 4 to about 20, from about 4 to about 16, or from about 4 to 8. In some embodiments, the N/P-ratio is above 1, about 1, about 2, about 3, about 4, about 5, about 6, about 7, or about 8. In certain embodiments, the RNA-LNPs have an N/P ratio of 8. In certain embodiments, the RNA-LNPs have an N/P ratio of 4. In certain embodiments, the RNA-LNPs have an N/P ratio of 2.

In some embodiments, the LNP comprises one or more mRNA molecules encoding an antigen (e.g., a viral antigen such as an influenza viral antigen, or a bacterial antigen).

LNPs can be prepared by various techniques presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion that results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in US 2011/0244026, US 2016/0038432, US 2018/0153822, US 2018/0125989, and US 2021/0046192 and can be used to practice the present disclosure. One exemplary process entails encapsulating mRNA by mixing it with a mixture of lipids, without first pre-forming the lipids into lipid nanoparticles, as described in US 2016/0038432. Another exemplary process entails encapsulating mRNA by mixing preformed LNPs with mRNA, as described in US 2018/0153822.

In one embodiment, nucleic acid is prepared in an aqueous buffer and mixed with an amphiphilic solution containing the lipid components of the LNPs. An amphiphilic solution for dissolving the four lipid components of the LNPs may be an alcohol solution. In some embodiments, the alcohol is ethanol. The aqueous buffer may be, for example, a citrate, phosphate, acetate, or succinate buffer and may have a pH of about 3.0-7.0, e.g., about 3.5, about 4.0, about 4.5, about 5.0, about 5.5, about 6.0, or about 6.5. The buffer may contain other components such as a salt (e.g., sodium, potassium, and/or calcium salts). In particular embodiments, the aqueous buffer has 1 mM citrate, 150 mM NaCl, pH 4.5.

In another embodiment, preformed LNPs are mixed with nucleic acid under conditions that allow formation of nucleic acid-LNPs in step a).

In one embodiment, the method comprises a step of synthesizing mRNA in vitro prior to step a). In one embodiment, the in vitro synthesized mRNA is purified prior to encapsulation in LNPs.

In one embodiment, encapsulation efficiency is at least 80%.

In some embodiments, the methods provided herein may further comprise a step of removing nucleic acid molecules which are not properly encapsulated in an LNP, but which are instead bound to the outer surface of the LNP. In particular, RNA molecules that are bound to the outer surface of LNPs may be removed by contacting RNA-LNPs with high salt. Thus, the methods provided herein may further comprise a step of dissociating RNA that is bound to the outer surface of LNPs. Specifically, the step of dissociating RNA may comprise contacting the RNA-LNPs with high salt prior to determining encapsulation efficiency. “High salt” as used herein may refer to salt that is provided at a final concentration ranging from 500 mM to 5 M. In some embodiments, salt is provided at a final concentration of 1 M to 5 M. In some embodiments, salt is provided at a final concentration of 0.75 M to 3 M. The salt may be NaCl. In some embodiments, NaCl is used at a final concentration ranging from about 500 mM to 5 M, optionally from 1 to 2 M.

In some embodiments, the step of dissociating the RNA that is bound to the outer surface of LNPs may additionally require heating. A suitable temperature for the dissociation step may be from about 60° C. to 95° C. In some embodiments, the dissociation step is performed at a temperature from about 70° C. to about 90° C. In some embodiments, the dissociation step is performed at a temperature from about 80° C. to 90° C. In some embodiments, the dissociation step is performed at a temperature of about 85° C.

The present invention further provides kits comprising various reagents and materials useful for carrying out inventive methods according to the present invention. The quantitative procedures described herein may be performed by diagnostic laboratories, experimental laboratories, or commercial laboratories. The invention provides kits which can be used in these different settings.

For example, materials and reagents for quantifying RNA encapsulation efficiency in a sample according to the methods provided herein may be assembled together in a kit. Each kit preferably comprises the reagents which render the procedure specific. In certain embodiments, a kit comprises two fluorophores, wherein a first fluorophore permeates LNPs and a second fluorophore does not permeate LNPs, such as those described herein. A kit optionally comprises additional reagents, such as a buffer, and instructions for using the kit according to a method of the invention.

The, the present disclosure further provides a kit for determining efficiency of RNA encapsulation in LNPs, comprising:
a first fluorophore that permeates LNPs;
a second fluorophore that does not permeate LNPs; and
optionally, instructions for use according to the method provided herein.

Kits or other articles of manufacture according to the invention may include one or more containers to hold various reagents. Suitable containers include, for example, bottles, vials, syringes (e.g., pre-filled syringes), ampules. The container may be formed from a variety of materials such as glass or plastic.

In some embodiments, kits of the present invention may include suitable control levels or control samples for determining control levels as described herein. For example, the kit may comprise RNA at a known concentration and/or LNPs having a known level of RNA encapsulation. In some embodiments, kits of the invention may include instructions for using the kit according to the method provided herein. In some embodiments, kits of the invention may further comprise instructions for RNA encapsulation in LNPs.

The present invention further relates to the use of Quant-iT™ HS in quantifying LNP-encapsulated RNA. The present invention further relates to the use of Quant-iT™ HS and RiboGreen® in quantifying LNP-encapsulated RNA. In one embodiment, the RNA is present at a final concentration of at least 0.25 μg/mL. In one embodiment, the RNA is present at a final concentration comprised between 0.25 μg/mL and 10 μg/mL.

The methods provided may be used in the quality control of LNP-encapsulated RNA and batch release of RNA-LNP compositions. Indeed, the present invention is particularly useful for quality control during manufacture of LNP-encapsulated mRNA and for characterization of LNP-encapsulated mRNA as an active pharmaceutical ingredient (API) in final therapeutic products.

The present invention comprises the following embodiments.

Embodiment 1. A method of determining the efficiency of RNA encapsulation in lipid nanoparticles (LNPs), comprising:
a) contacting a sample comprising RNA encapsulated in LNPs with a first fluorophore and a second fluorophore, thereby forming fluorophore-RNA complexes, and
b) detecting the fluorescence signals of the complexed first and second fluorophores,
   wherein the first fluorophore permeates the LNPs and wherein the second fluorophore does not permeate the LNPs.

Embodiment 2. The method of embodiment 1, wherein the first fluorophore is Quant-iT™ HS or SYBR® Green II.

Embodiment 3. The method of embodiment 1 or 2, wherein the second fluorophore is RiboGreen® or SYBR® Gold.

Embodiment 4. The method of any one of the preceding embodiments, wherein the RNA is from 10 to 50000 nucleotides in length.

Embodiment 5. The method of any one of the preceding embodiments, wherein the RNA is from 300 to 10000 nucleotides in length.

Embodiment 6. The method of any one of the preceding embodiments, wherein the RNA is from 500 to 5000 nucleotides in length.

Embodiment 7. The method of any one of the preceding embodiments, wherein the RNA is double-stranded RNA or single-stranded RNA.

Embodiment 8. The method of any one of the preceding embodiments, wherein the RNA comprises mRNA, miRNA, shRNA, rRNA, tRNA, snRNA, snoRNA, asRNA, siRNA, aiRNA, gRNA, and/or piRNA.

Embodiment 9. The method of any one of the preceding embodiments, wherein the first fluorophore and the second fluorophore are added to the sample simultaneously.

Embodiment 10. The method of any one of embodiments 1-8, wherein the first fluorophore and the second fluorophore are added to the sample sequentially.

Embodiment 11. The method of any one of the preceding embodiments, wherein RNA is present at a final concentration of at least 0.25 μg/mL, optionally at a final concentration comprised between 0.25 and 10 μg/mL.

Embodiment 12. The method of any one of the preceding embodiments, wherein the ratio of the first fluorophore to the second fluorophore is 2:1.

Embodiment 13. The method of any one of the preceding embodiments, wherein the first fluorophore is present at a final concentration of 0.5×.

Embodiment 14. The method of any one of the preceding embodiments, wherein the second fluorophore is present at a final concentration of 0.25×.

Embodiment 15. The method of any one of the preceding embodiments, wherein the method further comprises generating a standard curve for the first and second fluorescent signals.

Embodiment 16. The method of embodiment 15, wherein the method comprises determining the absolute amount of encapsulated RNA by matching each fluorescent signal detected in step b) with the corresponding standard curve.

Embodiment 17. The method of any one of embodiments 1-14, wherein the method further comprises determining the ratio of the second fluorescent signal to the first fluorescent signal.

Embodiment 18. A method of manufacturing LNPs encapsulating RNA, comprising:
a) encapsulating RNA in LNPs, and
b) determining efficiency of RNA encapsulation in LNPs according to the method of any one of embodiments 1-17.

Embodiment 19. The method of embodiment 18, wherein the RNA is mRNA.

Embodiment 20. The method of embodiment 19, wherein the method further comprises a step of synthesizing mRNA in vitro prior to step a).

Embodiment 21. The method of embodiment 20, wherein the in vitro synthesized mRNA is purified prior to encapsulation in LNPs.

Embodiment 22. The method of any one of embodiments 18-21, wherein lipids are mixed with RNA under conditions that allow formation of LNPs encapsulating RNA.

Embodiment 23. The method of any one of embodiments 18-22, wherein the encapsulation efficiency is at least 80%.

Embodiment 24. The method of any one of embodiments 18-23, wherein the method is conducted before releasing a batch of LNPs encapsulating RNA.

Embodiment 25. The method of any one of the preceding embodiments, wherein the LNPs comprise one or more ionizable lipids, one or more helper lipids, and one or more PEG-modified lipids.

Embodiment 26. A kit for determining efficiency of RNA encapsulation in LNPs, comprising:
a first fluorophore that permeates LNPs;
a second fluorophore that does not permeate LNPs; and
instructions for use according to the method provided herein.

Embodiment 27. Use of Quant-iT™ HS in quantifying LNP-encapsulated RNA.

In order that this invention may be better understood, the following examples are set forth. These examples are for purposes of illustration only and are not to be construed as limiting the scope of the disclosure in any manner.

### EXAMPLES

### Example 1: Materials & Methods

mRNA Production

mRNAs were produced as previously described (see Kalnin et al (2021), NPJ Vaccines 6(1):61 and US 2022/0142923). Briefly, mRNAs incorporating coding sequences were synthesized by in vitro transcription employing RNA polymerase with a plasmid DNA template encoding a desired gene using unmodified or modified nucleotides. An exemplary mRNA (mRNA 1) is about 2000 nucleotides in length. The resulting purified precursor mRNA was reacted further via enzymatic addition of a 5′ cap structure (Cap 1) and a 3′ poly(A) tail of approximately 200 nucleotides in length as determined by gel electrophoresis and purified. All mRNA preparations were analyzed for purity, integrity, and percentage of Cap 1 before storage at −80° C.

Encapsulation of mRNA in LNPs

For mRNA encapsulation in LNPs, an ethanolic solution of a mixture of lipids (cationic/ionizable lipid, helper lipid, cholesterol and polyethylene glycol-lipid) at a fixed lipid and mRNA ratio were combined with an aqueous buffered solution of target mRNA at an acidic pH under controlled conditions to yield a suspension of uniform LNPs. Upon ultrafiltration and diafiltration into a suitable diluent system, the resulting nanoparticle suspensions were diluted to final concentration, filtered, and stored frozen at −80° C. until use. The mRNA-LNP formulations were characterized for size by dynamic light scattering and encapsulation efficiency using the RiboGreen Assay. LNPs were composed of cationic lipid (40%), 1,2-dioleoyl-SN-glycero-3-phosphoethanolamine (DOPE; 30%), cholesterol (28.5%), and dimyristoyl-PEG2000 (DMG-PEG; 1.5%). Three types of LNP, each comprising a different cationic lipid (OF-02, cKK-E10, or GL-HEPES-E3-E12-DS-3-E14), were evaluated. Unless specified otherwise, LNPs comprise the OF-02 cationic lipid.

### Fluorescence Measurement

Fluorescence emissions (also referred to herein as fluorescence) were measured after a 20 min incubation of a sample in the presence of the fluorophore(s) in the dark at room temperature, unless otherwise indicated. Measurements were taken with a Spectramax 13 microplate reader (Molecular Devices) with the following settings:
RiboGreen: excitation 485 nm and emission 525 nm,
Quant-iT™ HS: excitation 644 nm and emission 673 nm,
SYBR® Gold: excitation 495 nm and emission 537 nm,
SYBR® Green 1l: excitation 495 nm and emission 520 nm,
SYTO® 17: excitation 621 nm and emission 646 nm.

In each case, excitation bandwidth was 9 nm and emission bandwidth was 15 nm.

### RiboGreen Assay (Comparative Example)

The standard RiboGreen assay was used to determine encapsulation efficiency, with samples tested in duplicate.

First, free mRNA and mRNA encapsulated in LNPs (mRNA-LNPs) were diluted to 2 ng/μL and 100 ng/μL mRNA, respectively, in TE buffer (10 mM Tris-HCl, 1 mM EDTA, pH 7.5). In parallel, mRNA-LNPs were diluted in TET buffer (TE buffer comprising 0.5% Triton X-100) to a final concentration of 2 ng/μL mRNA. Free mRNA and mRNA-LNP dilutions were performed in a black 96-well microplate with a flat clear bottom using 100 μL as the final volume. A standard scale of 8 independent dilutions of free mRNA (from 0 to 1 ng/μL) in TE or TET buffers was performed, with 4 independent dilutions of mRNA-LNPs at 20 ng/μL (from 0.5 to 20 ng/μL) in TE and 4 independent dilutions of mRNA-LNPs at 2 ng/μL (from 0.05 to 0.4 ng/μL) in TET.

RiboGreen® reagent was diluted to 1× in TE buffer extemporaneously and 100 μL of the 1× solution was added in each well, such that RiboGreen® was present at a final concentration of 0.5×. After incubation for 10 min in the dark at room temperature, RiboGreen® fluorescence was measured as described in Example 1. RNA quantification was performed based on a standard simple linear regression curve calculated from RiboGreen® fluorescence emissions of mRNA-LNPs in TE or TET buffers. The non-encapsulated mRNA concentration was determined by RiboGreen® fluorescence in mRNA-LNPs in TE buffer, and the total mRNA concentration by RiboGreen® emission in LNPs in TET buffer. Encapsulation efficiency was then calculated using the following equations:

$$\begin{matrix}Free mRNA ( \% ) = \frac{( RiboGreen emission in TET - corr esponding blank )}{( RiboGreen emission in TE - corresponding blank )} \\ Encapsulation efficiency ( \% ) = 100 - Free mRNA ( \% )\end{matrix}$$

### Evaluation of LNP Permeability to Fluorophores

To determine LNP permeability to fluorophores, mRNA-LNPs and corresponding free mRNA was diluted in TE at different total concentrations in 100 μL as final volume in a black 96-well microplate with a flat clear bottom. 100 μL of Quant-iT™ HS (2×) or 100 μL of SYTO® 17 (2 μM) were added in each well and samples were incubated for 20 min at room temperature in the dark. Fluorescence emissions were measured as described above. To determine if a fluorophore was able to permeate LNPs and specifically bound to RNA, the fluorescence standard curves were obtained and compared between free mRNA and mRNA-LNP ranges. SYTO® 17 was provided at a final concentration of 1 μM and Quant-iT™ HS was provided at a final concentration of 1×.

### Evaluation of a Dual Fluorescence Assay to Measure Encapsulation

Characteristics of two selected fluorophores are provided in Table 1, below.

TABLE 1
Characteristics of fluorophores used in the assay.

| | Excitation | Emission | Sensitivity |
|---|---|---|---|
| Fluorophore | Wavelength | Wavelength | scale |
| RiboGreen ® | 485 nm | 525 nm | 1-200 ng |
| Quant-iT ™ HS | 644 nm | 673 nm | 5-100 ng |

Quant-iT™ HS was coupled with RiboGreen®.

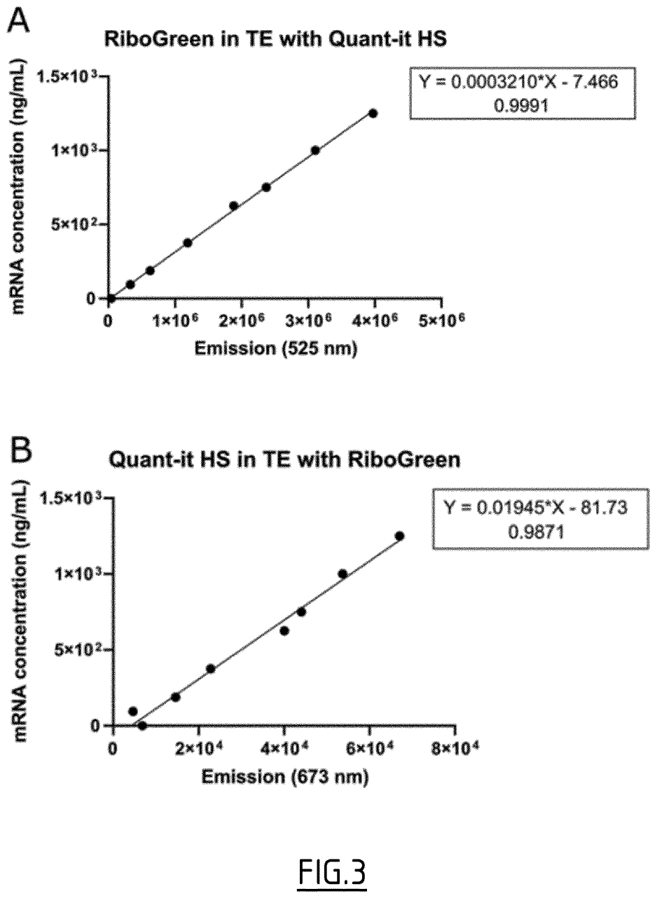

The assay was performed in duplicate for each point from an initial concentration of 2.5 ng/μL for free mRNA and 100 ng/μL for mRNA-LNPs in TE buffer. 8 independent dilutions of free mRNA (from 0 to 2.5 ng/μL) and 8 independent dilutions of mRNA-LNPs (from 0.5 to 20 ng/μL) were performed in a black 96-well microplate with a flat clear bottom using 100 μL as final volume. RiboGreen® and Quant-iT™ HS reagents were diluted extemporaneously to 0.5× and 1×, respectively, in TE buffer and 100 μL of the fluorophore mixture was added in each well, such that RiboGreen® and Quant-iT™ HS were present at a final concentration of 0.25× and 0.5×, respectively. Fluorescence was measured as described in Example 1. Standard simple linear regression curves were established for each fluorophore (FIG. 3).

RNA quantification was performed based on the two standard curves calculated from RiboGreen® and Quant-iT™ HS fluorescence using free mRNA in TE (see FIG. 3). Free mRNA concentration was then determined by measuring RiboGreen® fluorescence in a sample containing mRNA-LNPs, and the total mRNA concentration by measuring Quant-iT™ HS fluorescence in a sample containing mRNA-LNPs.

A summary of the fluorophore concentrations, final mRNA concentration ranges, and number of points measured for the RiboGreen assay or the assay of the present invention are provided in Table 2, below.

TABLE 2
Summary of fluorophore concentrations and free and encapsulated
mRNA ranges, with the number of data points of dilution analyzed.

| | | | Free mRNA samples | | | |
|---|---|---|---|---|---|---|
| | Fluorophore | | (control) | | mRNA-LNP Samples | |
| | | Quant-iT ™ | | Total mRNA | | Total mRNA |
| | RiboGreen ® | HS final | Number | conc. range | Number | conc. range |
| Assay | final conc. | conc. | of points | (μg/mL) | of points | (μg/mL) |
| RiboGreen | 0.5X | — | 16 | 0.025-0.5 | 8 | 1.25-10 |
| Assay | | | | (8 samples in TE | | (in TE), |
| | | | | and 8 samples in | | 0.025-0.2 |
| | | | | TET) | | (in TET) |
| Dual RNA | 0.25X | 0.5X | 8 | 0.094-1.25 | 8 | 0.25-10 |
| Encapsulation | | | | (in TE) | | (in TE) |
| Assay | | | | | | |

Free mRNA dilutions of known concentration were used to establish a standard curve needed to quantify free and total mRNA concentrations in the samples. Conc.: concentration, TE: Tris-EDTA, TET: Tris-EDTA-Triton.

### Use of the Dual RNA Encapsulation Assay in High-Throughput Screening

The assay was performed in triplicate for each point using mRNA-LNP samples at an initial concentration of 20 ng/μL in TE buffer. Two independent dilutions of mRNA-LNP samples (to 2 and 2.5 ng/μL) were performed in TE buffer in a black 96-well microplate with flat clear bottom with 100 μL as final volume. A blank without mRNA-LNP was also included to remove background fluorescence. RiboGreen® and Quant-iT™ HS reagents were diluted to 0.5× and 1× respectively in TE buffer extemporaneously and 100 μL of the fluorophore mixture was added in each well. RiboGreen® and Quant-iT™ HS emissions were measured as described above.

Encapsulation efficiency was determined using the following equations:

$$\begin{matrix}Free mRNA ( \% ) = \frac{( RiboGreen E mission in mRNA - LNPs - corr esponding blank )}{( Quant iT HS E mission in mRNA - LNPs - corresponding blank )} \\ Encapsulation efficiency ( \% ) = 100 - Free mRNA ( \% )\end{matrix}$$

### Example 2: Fluorophore Selection

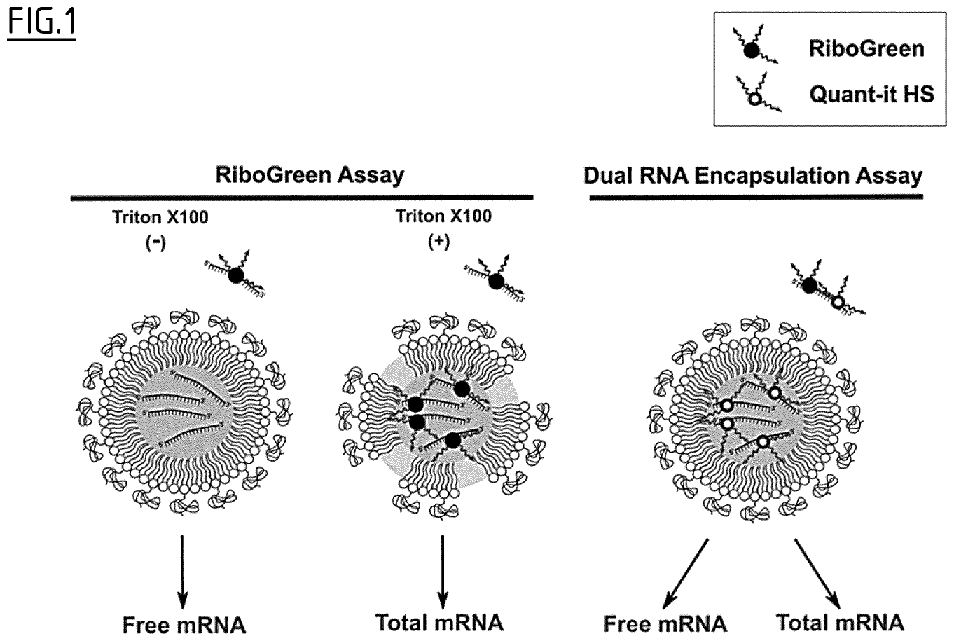

The use of two fluorophores in a single well to determine RNA encapsulation efficiency (also referred to herein as a ‘dual RNA encapsulation assay’) was evaluated. An illustration of the principle of the method, as compared to the RiboGreen assay, is provided in FIG. 1.

Initially, compatible fluorophores were selected for use in the assay. To be used in the same well, the two selected fluorophores should not show a spectral overlap (i.e., in excitation and emission wavelengths). The two selected fluorophores should also detect similar mRNA concentration ranges (i.e., have a similar sensitivity scale). This is notably the case for RiboGreen® and Quant-iT™ HS (see Table 1 above).

RiboGreen® does not permeate LNPs, given that in the RiboGreen assay a detergent must be added to lyse LNPs in view of measuring fluorescence of total RNA emissions. Thus, the second fluorophore should be able to permeate LNPs.

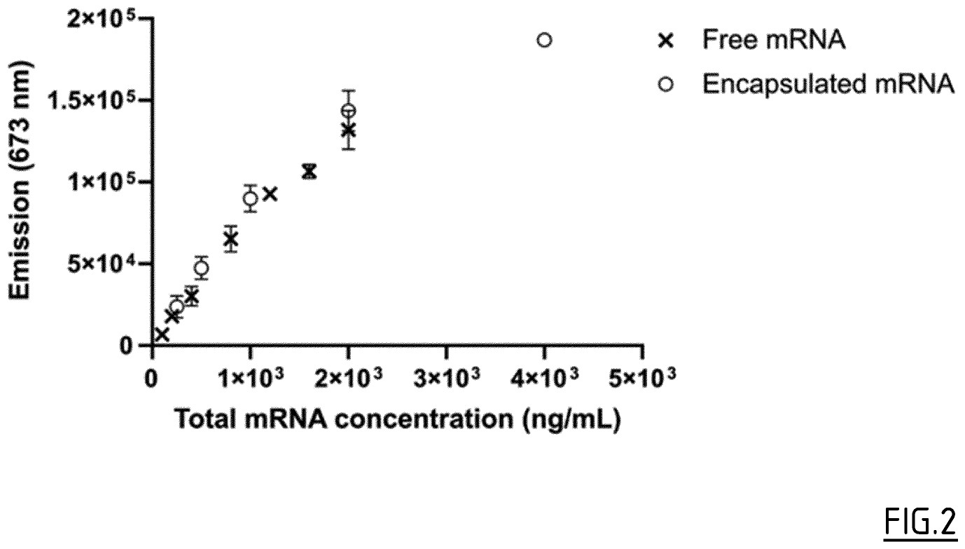

The emissions of Quant-iT™ HS were measured in the presence of free mRNA or encapsulated mRNA (i.e., mRNA-LNPs) to determine its ability to label total mRNA in LNPs. Quant-iT™ HS emissions were the same between free and encapsulated mRNA demonstrating the permeability properties of this fluorophore as concerns LNPs (see FIG. 2). These results further illustrate that Quant-iT™ HS does not interact with lipids comprising the LNPs.

The two fluorophores should also not show any binding to lipids or competition with one another. The quantification of non-encapsulated or total mRNA concentrations is based on a standard free mRNA curve, which must fit with a standard linear regression. RiboGreen® and Quant-iT™ HS were thus tested in the same well for their ability to concomitantly label free mRNA at different concentrations (see FIG. 3). Results showed that emissions from both fluorophores resulted in linear regressions that could be used as standard curves. Moreover, neither RiboGreen® nor Quant-iT™ HS lost their sensitivity range in the presence of the other. Thus, RiboGreen® and Quant-iT™ HS were the two fluorophores selected to further evaluate the dual labeling assay.

### Example 3: Evaluation of the Dual RNA Encapsulation Assay

To validate the assay according to the invention, it was performed in parallel to the RiboGreen assay using the fluorophore concentrations and standard mRNA and LNP ranges provided in Table 2. As shown in Table 3, below, encapsulation efficiency was highly similar between the two methods. Thus, the method of the invention can be successfully used to determine encapsulation efficiency. In addition, the invention is advantageous as the variability is reduced compared to RiboGreen assay, as shown in Table 3.

TABLE 3
Encapsulation efficiency as determined with the RiboGreen
assay or the assay of the present invention.

| | Encapsulation | Total mRNA | Free mRNA |
|---|---|---|---|
| Assay | Efficiency (%) | conc. (μg/mL) | conc. (μg/mL) |
| RiboGreen Assay | 95 | 1007 ± 17% | 51 ± 53% |
| Dual RNA | 96 | 1003 ± 11% | 40 ± 26% |
| Encapsulation Assay | | | |

The RiboGreen assay and the dual RNA encapsulation assay were performed as described above using unmodified free mRNA and mRNA encapsulated in LNPs comprising the OF-02 cationic lipid. mRNA concentration was determined based on the standard linear regression lines established for each assay. Standard deviation (%) was calculated for both mRNA concentrations from 4 independent dilution points of mRNA-LNPs in triplicate.

### Example 4: Determination of the Range of Encapsulation Efficiency that can be Measured with the Dual RNA Encapsulation Assay

The ability of the dual RNA encapsulation assay to detect different levels of encapsulation efficiency was evaluated. As illustrated in Table 4, below, the assay of the invention was able to accurately quantify both free and total mRNA concentrations in samples comprising LNPs, without the addition of detergent, in contrast to what is required in the classic RiboGreen assay. The determination of encapsulation efficiency was accurate at various encapsulation rates (here, as low as 50%).

TABLE 4
Different encapsulation efficiencies of as determined
by the dual RNA encapsulation assay.

| Encapsulation | | Total mRNA | | Free mRNA | |
|---|---|---|---|---|---|
| Efficiency (%) | | conc. (μg/mL) | | conc. (μg/mL) | |
| Expected | Measured | Expected | Measured | Expected | Measured |
| 95 | 96 | 1000 | 1007 | 50 | 43 |
| 70 | 69 | 573 | 571 | 172 | 177 |
| 50 | 45 | 419 | 371 | 210 | 205 |

The dual RNA encapsulation assay was performed as described above using unmodified free mRNA encapsulated in LNPs comprising the OF-02 cationic lipid. A source sample known to have a total mRNA concentration of 1000 μg/mL and an encapsulation efficiency of 95% (as determined by the RiboGreen assay) was used as a baseline. Free mRNA was added to the sample to artificially modulate encapsulation efficiency to 70% and 50%. The dual RNA encapsulation assay uses a standard linear regression line calculated from the labeling of free mRNA by both the RiboGreen® and Quant-iT™ HS fluorophores. The non-encapsulated (free) and total mRNA concentrations are then determined respectively by RiboGreen® and Quant-iT™ HS fluorescence. Finally, encapsulation efficiency was determined by the ratio between the concentration of free mRNA and total mRNA. 4 independent dilution points of mRNA-LNPs were measured for each concentration in triplicate.

### Example 5: Establishment of a High-Throughput Dual RNA Encapsulation Assay

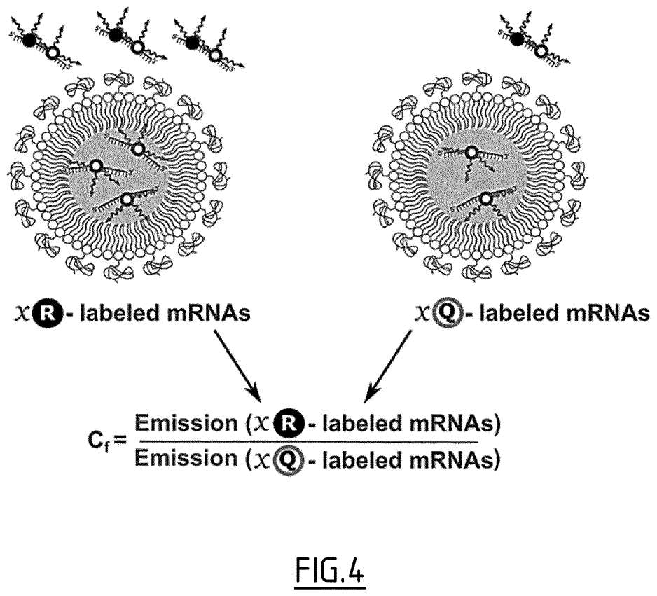

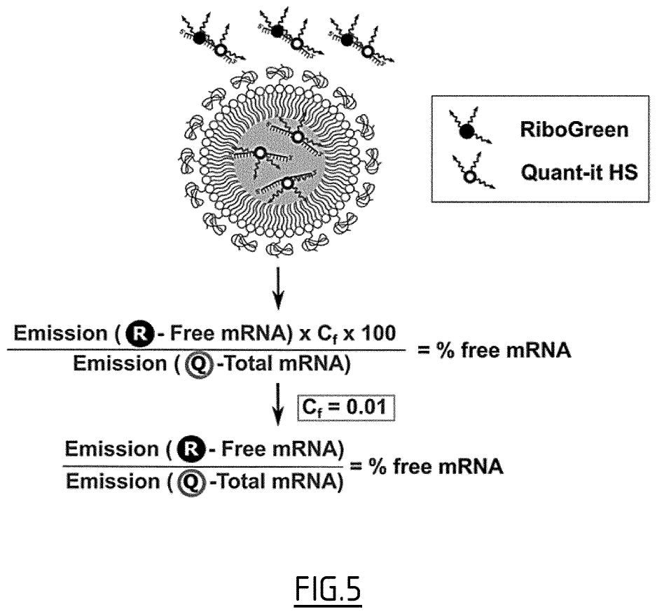

A protocol for high-throughput determination of encapsulation efficiency, which does not require the measurement of mRNA concentrations, was developed. This method compares the ratio of fluorophore emissions in the sample to directly determine the rate of encapsulation. First, the coefficient of fluorescence (Cf) between RiboGreen® and Quant-iT™ HS was determined for the same concentration of labeled mRNA (FIG. 4). Different concentrations of non-encapsulated mRNA and their corresponding identical concentrations of total mRNA were labeled with the fluorophores and the ratio of emissions were calculated (Table 5). Independently of mRNA concentration, the ratio was always 0.01. The consistency of the Cr allowed for its integration into a new encapsulation efficiency equation, where the percentage of non-encapsulated mRNA was given by the simple ratio of RiboGreen® emission to Quant-iT HS emission in the same well (FIG. 5).

TABLE 5
Coefficient of fluorescence between the RiboGreen ® and
the Quant-iT ™ HS fluorophores for the
same amount of labeled mRNA.

| Labeled mRNA conc. (ng/mL) | C_{f}(RiboGreen ®/Quant-iT ™ HS) |
|---|---|
| 5000 | 0.01 |
| 2500 | 0.01 |
| 1250 | 0.01 |
| 500 | 0.01 |
| 250 | 0.01 |

LNPs encapsulating unmodified mRNA were diluted at different rates and the mRNA labeled with both the RiboGreen® and Quant-iT™ HS fluorophores. The Coefficient of Fluorescence (Cf) between RiboGreen® and Quant-iT™ HS fluorescence emissions was determined between conditions where the free mRNA concentration (RiboGreen® fluorescence) was the same as the total mRNA concentration (Quant-iT™ HS fluorescence) (N=3).

A protocol was then designed to use two dilution points of mRNA-LNPs which correspond to the more precise range of sensitivity for both fluorophores (final mRNA concentration of 1000 and 1250 ng/mL, respectively, in the diluted samples; see Table 6). The accuracy of this high-throughput assay was validated for different levels of encapsulation efficiency (see Table 7).

TABLE 6
Standard and Dual RNA Encapsulation assays’ biological materials.

| | | | Free mRNA | | mRNA-LNPs | |
|---|---|---|---|---|---|---|
| | Fluorophores | | | Total mRNA | | Total mRNA |
| | RiboGreen ® | Quant-iT ™ | Number | conc. range | Number | conc. range |
| Assay | conc. | HS conc. | of points | (μg/mL) | of points | (μg/mL) |
| RiboGreen | 0.5X | — | 16 | 0.025-0.5 | 8 | 1.25-10 |
| Assay | | | | (8 in TE and 8 in | | (in TE) |
| | | | | TET) | | 0.025-0.2 |
| | | | | | | (in TET) |
| Dual RNA | 0.25X | 0.5X | 0 | — | 2 | 1 and 1.25 |
| Encapsulation | | | | | | (in TE) |
| Assay | | | | | | |

The table summarizes fluorophore concentrations and the free and encapsulated mRNA (LNP) ranges used to quantify free and total mRNA concentrations in the mRNA-LNP samples. High-throughput (HT) screening based on the dual RNA encapsulation protocol provided in Example 4, but without absolute quantification of mRNA concentration, was performed.

TABLE 7
Encapsulation efficiency of mRNA-LNPs using a simplified assay.

| Expected | | Ratio (RiboGreen ® | | Measured |
|---|---|---|---|---|
| encapsulation | Total mRNA | Em/Quant-iT ™ | | encapsulation |
| efficiency (%) | conc. (ng/mL) | HS Em) | | efficiency (%) |
| 95 | 1250 | 4.37 | 3.76 | 96 ± 0.32% |
| | 1000 | 4.43 | 4.40 | |
| 50 | 1250 | 54.34 | 48.08 | 49 ± 3.8% |
| | 1000 | 46.95 | 53.67 | |

The mRNA-LNP source sample is known to have an encapsulation efficiency of 95%. Free mRNA was added to the sample to artificially modulate encapsulation efficiency to 50%. LNPs were diluted to 2000 and 2500 ng/mL of total RNA before labeling free and total mRNA with the RiboGreen® and Quant-iT™ HS fluorophores, respectively. The percentage of free mRNA was determined by the ratio between RiboGreen® and Quant-iT™ HS fluorescence (N=3). The experiment was performed as two independent dilutions and standard deviations of encapsulation efficiencies were calculated from both dilutions. Em: fluorescence emission.

As illustrated here, the determination of encapsulation efficiency is highly accurate even when only two dilution points are used. Thus, the method of the invention can be successfully used in high throughput applications, such as screening, where fully quantitative methods are not required.

### Example 6: Evaluation of Encapsulation Efficiency with Various mRNA-LNPs

Various mRNAs (comprising unmodified or modified nucleotides) and LNPs comprising different cationic lipids were evaluated using the high-throughput method described in Example 5. Results obtained with this method were similar to those obtained with the RiboGreen assay and indicate that the method may be used regardless of the LNP composition, the mRNA sequence, or the presence of modified nucleosides in the mRNA (see Table 8).

TABLE 8
Encapsulation efficiency as determined by the RiboGreen assay
and the dual RNA encapsulation assay of the invention.

| | | | Encapsulation | |
|---|---|---|---|---|
| | | | Efficiency (%) | |
| | | Modified | | Dual RNA |
| Cationic | | mRNA | RiboGreen | encapsulation |
| lipid | mRNA | nucleosides | Assay | Assay |
| OF-02 | 1 | no | 95 | 96 ± 0.1% |
| cKK-E10 | 1 | no | 83 | 80 ± 3% |
| GL-HEPES-E3- | 2 | yes | 94 | 92 ± 1% |
| E12-DS-3-E14 | | | | |
| GL-HEPES-E3- | 3 | yes | 94 | 92 ± 2% |
| E12-DS-3-E14 | | | | |
| GL-HEPES-E3- | 4 | yes | 95 | 92 ± 1% |
| E12-DS-3-E14 | | | | |

The encapsulation efficiency of mRNA in 5 different (comprising 3 different cationic lipids and 4 different mRNAs with or without chemically modified nucleosides) were determined in parallel. The dual RNA encapsulation assay uses the ratio of the RiboGreen® (non-encapsulated mRNA) and the Quant-iT™ HS (total mRNA) emissions in the same well (N=3). The standard deviation of encapsulation efficiency was calculated from two different LNP dilutions as described in Example 5 (see Table 6).

### Example 7: Evaluation of Alternative Fluorophores

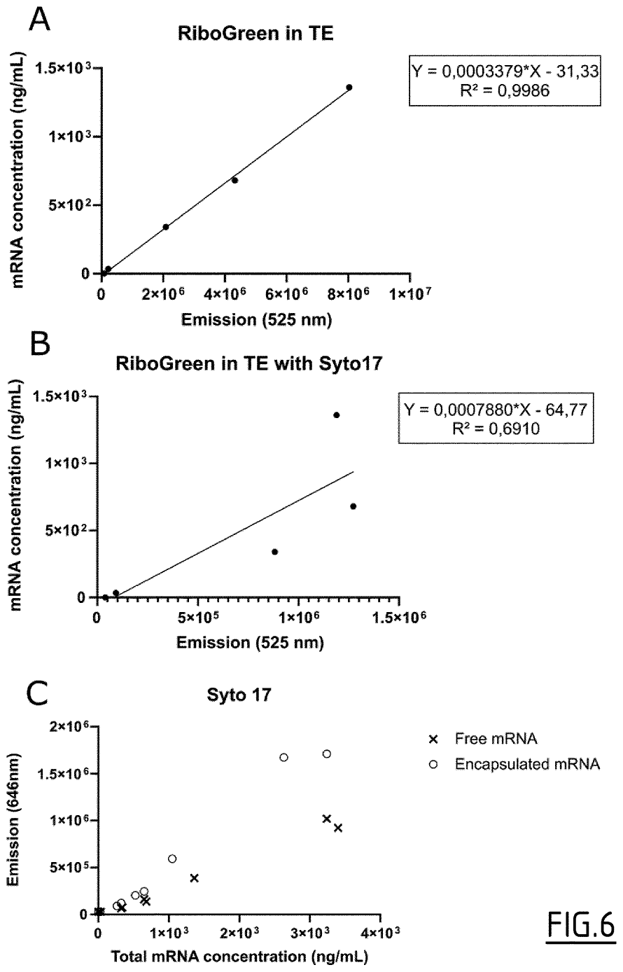

SYTO® 17 was evaluated to determine if it may be used as an alternative fluorophore in the method of the invention. While a linear standard curve is obtained with RiboGreen® alone, this is no longer the case when RiboGreen® is combined with SYTO® 17 (FIG. 6., compare panels A and B). This indicates that RiboGreen® fluorescence is modified by the presence of SYTO® 17. Furthermore, as shown in FIG. 6C, the standard curve established for SYTO® 17 differed for free mRNA and mRNA-LNPs. In particular, fluorescence is higher for mRNA-LNPs than with free mRNA. Without being bound by theory, this suggests that SYTO® 17 may interact with a lipid component of the LNPs.

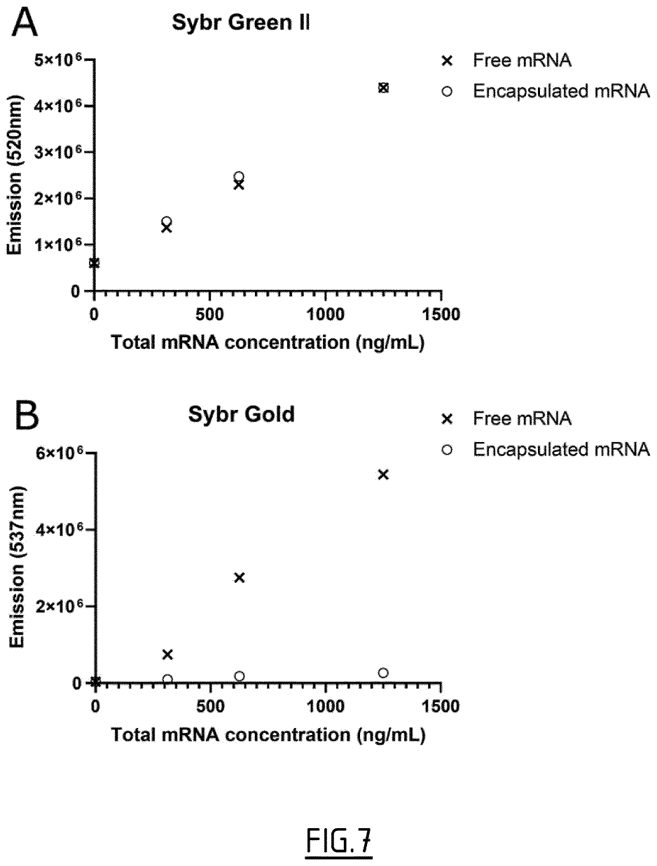

SYBR® Gold and SYBR® Green II were also evaluated to determine if they may be used as alternative fluorophores. As illustrated in FIG. 7, the standard curve obtained for free mRNA and encapsulated mRNA were identical when using SYBR® Green 1l, indicating that this fluorophore permeates LNPs. In contrast, the standard curve obtained for SYBR® Gold varied according to the sample tested (i.e., free mRNA only or mRNA-LNPs). In particular, almost no fluorescence emissions were detected when mRNA-LNPs were contacted with SYBR® Gold. This suggests that this fluorophore does not permeate LNPs.

Overall, the simple linear regression lines determined when labeling mRNA with SYBR® Green II or SYBR® Gold indicates that either of these fluorophores may be utilized in the dual RNA encapsulation assay in combination with compatible fluorophores having detectably distinct fluorescence emissions.

### Example 8: Automation of Dual RNA Encapsulation Assay and Validation of the Method with Two Different Spectrophotometers

The dual RNA encapsulation assay was implemented on a Starlet Hamilton robotic platform in view of automating the method. A source sample comprising unmodified mRNA encapsulated in LNPs comprising the OF-02 cationic lipid known to have a total mRNA concentration of 1000 μg/mL and an encapsulation efficiency of 95% (as determined by the RiboGreen assay described in Example 1 above) was used. Unmodified free mRNA was added to the source sample to artificially modulate encapsulation efficiency to 70% and 50%. Various concentrations of these mRNA-LNPs (ranging from 20 to 200 ng/μL) were loaded in a PCR 96-well plate using 40 μL as final volume. Samples were diluted to 10 ng/μL with TE buffer as necessary in another PCR 96-well plate. Finally, samples were diluted in a black 384-well microplate with a flat clear bottom to provide two data points per sample, loaded in duplicate (i.e., a total of four replicates per condition). RiboGreen® and Quant-iT™ HS reagents were diluted extemporaneously by the robot to 0.5× and 1×, respectively, in TE buffer and 40 μL of the fluorophore mixture was added in each well, such that RiboGreen® and Quant-iT™ HS were present at a final concentration of 0.25× and 0.5×, respectively. Fluorescence was measured as described in Example 1 using either a Cytation 7 (Agilent Biotek) or Spectramax i3 spectrophotometer, and encapsulation efficiency was then determined using the corresponding equation provided in Example 1. All steps, from initial dilution of the samples to measurement of fluorescence by an appropriate spectrophotometer, were performed on the robotic platform.

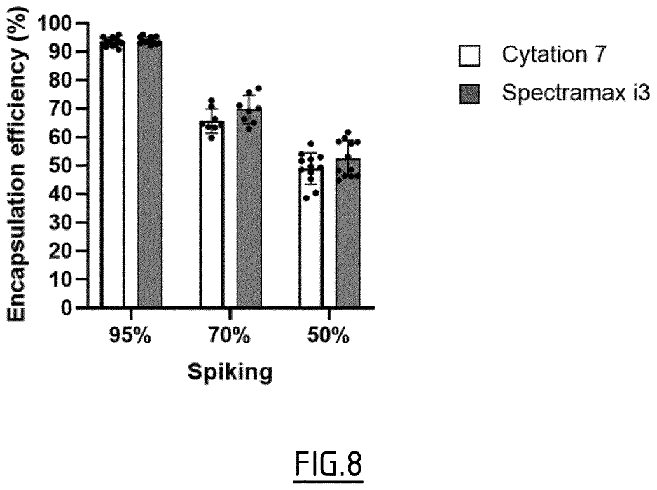

The results shown in FIG. 8 demonstrate that the method is automatable. Furthermore, measurements are accurate independent of the spectrophotometer used.

### CONCLUSIONS

In view of the above, the method of the invention represents an improved method of determining encapsulation efficiency. In particular, the number of samples required is reduced by at least 2-fold, as dual measurements are able to be determined from a single well. The method can be used in both quantitative and high-throughput screening approaches and shows a high level of accuracy in determining encapsulation efficiency of nucleic acid in LNPs. In particular, by using the ratiometric approach provided in Example 5 for measurement of LNP-RNA encapsulation efficiency, many different RNAs can be evaluated without the need to provide a standard curve for each sample for absolute quantification of free and encapsulated mRNA. Furthermore, using the automated dual RNA encapsulation assay of example 8, the protocol is fully automated with up to 95 samples processed in less than two hours and requires only limited amounts of raw materials (e.g. mRNA-LNP samples, fluorophores).

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

1. Method of determining the efficiency of RNA encapsulation in lipid nanoparticles (LNPs), comprising:
a) contacting a sample comprising RNA encapsulated in LNPs with a first fluorophore and a second fluorophore, thereby forming fluorophore-RNA complexes, and
b) detecting the fluorescence signals of the complexed first and second fluorophores,
wherein the first fluorophore permeates the LNPs and wherein the second fluorophore does not permeate the LNPs.

2. The method of claim 1, wherein the first fluorophore is Quant-iT™ HS or SYBR® Green II.

3. The method of claim 1, wherein the second fluorophore is RiboGreen® or SYBR® Gold.

4. The method of claim 1, wherein the RNA is from 10 to 50000 nucleotides in length.

5. The method of claim 1, wherein the RNA is double-stranded RNA or single-stranded RNA.

6. The method of claim 1, wherein the RNA comprises mRNA, miRNA, shRNA, rRNA, tRNA, snRNA, snoRNA, asRNA, siRNA, aiRNA, gRNA, and/or piRNA.

7. The method of claim 1, wherein the first fluorophore and the second fluorophore are added to the sample simultaneously.

8. The method of claim 1, wherein the ratio of the first fluorophore to the second fluorophore is 2:1.

9. The method of claim 1, further comprising determining the ratio of the second fluorescent signal to the first fluorescent signal.

10. The method of claim 1, further comprising generating a standard curve for the first and second fluorescent signals.

11. The method of claim 10, further comprising determining the absolute amount of encapsulated RNA by matching each fluorescent signal detected in step b) with the corresponding standard curve.

12. A method of manufacturing LNPs encapsulating RNA, comprising
a) encapsulating RNA in LNPs, and
b) determining efficiency of RNA encapsulation in LNPs according to the method of claim 1.

13. The method of claim 12, wherein the method further comprises a step of synthesizing mRNA in vitro prior to step a).

14. A kit for determining efficiency of RNA encapsulation in lipid nanoparticles (LNPs), comprising:
a first fluorophore that permeates LNPs;
a second fluorophore that does not permeate LNPs; and
instructions for use according to the method of claim 1.

15. Use of Quant-iT™ HS in quantifying LNP encapsulated RNA.
